(19) United States
(12) Patent Application Publication
(10) Pub. No.: US 2025/0111943 A1
(43) Pub. Date: Apr. 3, 2025

(54) Method for Evaluating Biomarkers in a Biological Sample

(71) Applicant: Thriva Limited, London (GB)

(72) Inventors: Thomas James Livesy, London (GB); Myles Philip Cowper-Coles, London (GB); Steven Michael Pewsey, London (GB); Emma Elizabeth Dinneen, London (GB)

(21) Appl. No.: 18/374,004

(22) Filed: Sep. 28, 2023

(51) Int. Cl. G16H 50/30; G16B 20/00

(52) U.S. Cl. CPC G16H 50/30; G16B 20/00

(57) ABSTRACT

The invention relates to a computer implemented method of automatically evaluating biomarkers in a biological sample taken from a subject. The method comprises receiving analytical data relating to the biological sample. Biomarkers identified in the analytical data are processed to determine an escalation level or score. This is achieved by retrieving a rule for each of the biomarkers identified in the analytical data, each rule defining a set of thresholds corresponding to some or all of a set of escalation levels, the escalation levels being indicative of different respective levels of a need to escalate the results to a third party; comparing values of said biomarkers to the thresholds defined by their respective rules to determine respective escalation levels for each of said biomarkers; and determining, selecting or calculating an overall or final escalation level based on the determined respective escalation levels.

### FIELD OF THE INVENTION

The invention relates to the field of medical diagnosis, and particularly, but not exclusively, to the field of medical technology for evaluating biomarkers in a biological sample. The method relates particularly, but not exclusively, to evaluating biomarkers in a bodily fluid sample such as, for example, a blood sample or a urine sample in order to automatically determine, select, or calculate an escalation level for a subject from which the biological sample was obtained.

### BACKGROUND OF THE INVENTION

In a general clinical setting, a subject or a patient may often be required to conduct different medical tests to identify or to confirm a health issue or a potential health issue. Many common medical tests are based on body fluid samples obtained from the subject. For example, a blood test can provide important information about the subject's overall health, as well as assist in diagnosing and monitoring a wide range of medical conditions such as infections, anemia, heart, liver, or kidney problems, etc. Nowadays, various types of blood tests can be performed and the specific tests required may depend on the subject's medical needs, health conditions, and medical history.

When a blood test is required to diagnose a patient's condition, a doctor may order a blood test for the patient by generating a requisition to specify the type of test or tests to be performed. The patient's blood sample is sent to a laboratory or testing facility. In the laboratory, trained technicians will process the sample and perform the requested tests using specialized equipment and techniques. Results of the tests are then recorded and sent back to the doctor who ordered them for manual review and evaluation. The doctor will review and interpret the test results based on the doctor's medical knowledge in the context of the patient's medical history and current symptoms. They will determine whether the results are normal or abnormal, and decide whether further testing or treatment is necessary. If the doctor has any questions or concerns about the test results, they may contact the laboratory to discuss the findings. In some cases, the laboratory may detect abnormalities or unexpected results in the blood sample that may require further testing or analysis. If this happens, the laboratory may contact the requisitioning doctor to discuss the results and determine next steps. Otherwise, the doctor may contact the patient to discuss his observations and suggestions. Overall, the process of ordering, testing, and analyzing test results for medical diagnosis involves close collaboration between the patients, doctors, and laboratory professionals to ensure that patients receive accurate results, and the time it may take will depend on the type of tests and the level of review and interpretation required.

In light of the Covid pandemic, there is much greater pressure on medical services than was previously the case. There is therefore a need to more efficiently and effectively assess test data obtained from subjects' biological samples.

### OBJECTS OF THE INVENTION

An object of the present invention is to provide a method, system, or device for evaluating biomarkers in a biological sample.

Another object of the present invention is to provide an automated or partially automated method of evaluating biomarkers in a biological sample taken from a subject to determine, select, or calculate an escalation level for the subject.

A further object of the present invention is to mitigate or obviate to some degree one or more problems associated with known methods, systems, or devices for processing and evaluating biomarkers in a biological sample.

The above objects are met by the combination of features of the main claims; the sub- claims disclose further advantageous embodiments of the invention.

One skilled in the art will derive from the following description of other objects of the invention. Therefore, the foregoing statements of the object are not exhaustive and serve merely to illustrate some of the many objects of the present invention.

### SUMMARY OF THE INVENTION

The invention relates to a computer implemented method of automatically evaluating biomarkers in a biological sample taken from a subject. The method comprises receiving analytical data relating to the biological sample. Biomarkers identified in the analytical data are processed to determine an escalation level or score. This is achieved by retrieving a rule for each of the biomarkers identified in the analytical data, each rule defining a set of thresholds corresponding to some or all of a set of escalation levels, the escalation levels being indicative of different respective levels of a need to escalate the results to a third party; comparing values of said the biomarkers to the thresholds defined by their respective rules to determine respective escalation levels for each of said biomarkers; and determining, selecting or calculating an overall or final escalation level based on the determined respective escalation levels.

In a first main aspect, the invention provides a computer implemented method of automatically evaluating biomarkers in a biological sample taken from a subject, comprising the steps of: receiving analytical data relating to a plurality of analytes identified from the biological sample; processing the received analytical data against a predetermined set of biomarkers to automatically determine an escalation level for the subject by: retrieving a rule for each of the biomarkers identified in the analytical data, each rule defining a set of thresholds corresponding to some or all of a set of escalation levels, the escalation levels being indicative of different respective levels of a need to escalate the results; comparing values of said biomarkers to the thresholds defined by their respective rules to determine respective escalation levels for each of biomarkers; and determining, selecting or calculating an overall escalation level based on the determined respective escalation levels for each of said at least some of the biomarkers.

The set of escalation levels is preferably common to all of the rules.

In a second main aspect, the invention provides a system for escalating test results for a subject by implementing the steps according to the first main aspect. The system comprises a receiving module for receiving analytical data relating to one or more analytes identified from a biological sample obtained from the subject; and a processor for processing the received analytical data against a predetermined set of biomarkers to thereby automatically determine a need to escalate the results.

In a third main aspect, the invention provides a device for evaluating biomarkers in a biological sample taken from a subject. The device comprises a processor adapted to process a received analytical data relating to one or more analytes identified from the biological sample, the analytical data being processed against a predetermined set of biomarkers to determine a need to escalate the results.

The summary of the invention does not necessarily disclose all the features essential for defining the invention; the invention may reside in a sub-combination of the disclosed features.

### BRIEF DESCRIPTION OF THE DRAWINGS

The foregoing and further features of the present invention will be apparent from the following description of preferred embodiments which are provided by way of example only in connection with the accompanying figure, of which:

FIG. 1 is a block diagram showing one embodiment of the system according to the present invention;

FIG. 2 is a flow diagram showing a method according to the present invention;

FIG. 3 comprises a system context diagram illustrating the relationship of the escalation level calculator with other modules/components of the escalation level system in accordance with the invention;

FIG. 4 is a more detailed view of the escalation level system of FIG. 3 showing the preferred software containerization of the system; and

FIG. 5 is a schematic diagram of the modules/components comprising the escalation level calculator in accordance with the invention.

### DESCRIPTION OF PREFERRED EMBODIMENTS

The following description is of preferred embodiments by example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearance of the phrase “in one embodiment” in various specifications does not necessarily refer to the same embodiment, nor are separate or alternative embodiments mutually exclusive of other embodiments. Moreover, various features are described, which may be exhibited by some embodiments and not by others. Similarly, various requirements are described, which may be requirements for some embodiments but not other embodiments.

It should be understood that the elements shown in the figures may be implemented in various forms of hardware, software, or combinations thereof. Preferably, these elements are implemented in a combination of hardware and software on one or more appropriately programmed general-purpose devices, which may include a processor, a memory and input/output interfaces.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

15 Thus, for example, it will be appreciated by those skilled in the art that the block diagram presented herein represent conceptual views of the system and the device embodying the principles of the invention.

The functions of the various elements described or shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

In the claims hereof, any element expressed as a means for performing a specified function is intended to encompass any way of performing that function including, for example, a) a combination of circuit elements that performs that function or b) software in any form, including, therefore, firmware, microcode, or the like, combined with appropriate circuitry for executing that software to perform the function. The invention as defined by such claims resides in the fact that the functionalities provided by the various recited means are combined and brought together in the manner which the claims call for. It is thus regarded that any means that can provide those functionalities are equivalent to those shown herein.

In the context of the present invention, the terms “computer, “computer based”, “computer device”, “computer implemented”, “processor”, “processing device” are intended to encompass any suitable processing device. For example, any components, systems, or devices described herein can be associated with any computer or processing devices such as general-purposed computers, client terminals, or other suitable devices such as mobile phones, smart phones, tablets, smart watches, or any other mobile computing devices.

The present invention relates to a computer implemented method, system, and/or device for automatically evaluating biomarkers in a biological sample taken from a subject, and particularly but not exclusively, for evaluating a level of risk or a level of a need to escalate results by automatically processing test results based on a biological sample of the subject such as the subject's body fluids. In one embodiment, the biological sample may include, but is not limited to, a blood sample.

The present invention compares the test results on analytes from the subject's blood sample with a preferably predetermined or defined set of biomarkers, and automatically assigns a category of risk level (escalation level) to each of the matched biomarkers based on a corresponding set of predetermined thresholds or rules. The thresholds are determined by clinical professionals based on authoritative medical literatures, such as the Royal College of Pathology Critical Result Guidelines, National Best Practice Guidelines, and other existing clinical literatures. In one embodiment, the assigned levels of risk may include, but are not limited to, the following escalation categories: none/no, very low, low, medium, high, and very high risks in respect of the tested analytes and biomarkers. The assigned level of risk may subsequently be used to determine the possible course of action that the clinicians and/or doctors may recommend to the test subject or patient in view of the health or medical conditions. For example, the test subject may be advised to consult their general practice doctor within 7 days if a medium level of risk is determined; to consult their general practice doctor within 3 days if a high level of risk is determined; or to seek immediate medical assistance within 24 hours if a very high level of risk is determined. The present invention is therefore advantageous in that it automates the review and evaluation processes of the test results which currently requires manual review and interpretation by medical doctors or clinical professionals. The users of the present invention, which can be the test subjects or patients, the clinicians, hospital professionals initiating or conducting the blood tests, and/or the subjects' doctors can be timely informed and alerted such that follow up actions can be implemented.

One technical problem addressed by the present invention is how to provide or assign a score, value or level using a defined scale, defined set of levels or the like to quantitatively and qualitatively different thresholds or sets of thresholds for unrelated or loosely related biomarkers in a subject's biological sample results.

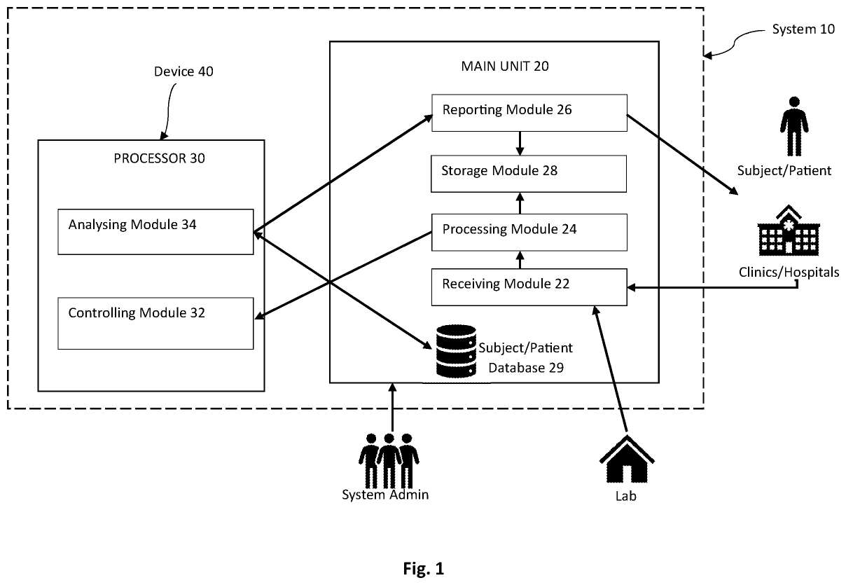

Referring to FIG. 1, shown is a block diagram of a system 10 according to one embodiment of the present invention. The system 10 may comprise a single, integrated device or unit comprising the various modules for implementing the claimed functional steps. Alternatively, the system 10 can be configured to comprise separate functional blocks as individual modules for performing different functions of the present invention. For example, in the embodiment as shown in FIG. 1, the system 10 may comprise a main unit 20 such as an online platform to receive information from and to report information to the users, which may include, but is not limited to, patients, test subjects, system administrators, professionals from medical clinics or hospitals, and/or laboratory technicians, etc. In one embodiment, the main unit 20 can be provided or configured with different user terminals or interfaces for the users to communicate with the main unit 20. For example, the user interfaces may include interfaces such as a web-based platform and/or a website operating at different computing devices, and/or an application (APP) operating at mobile devices or the like.

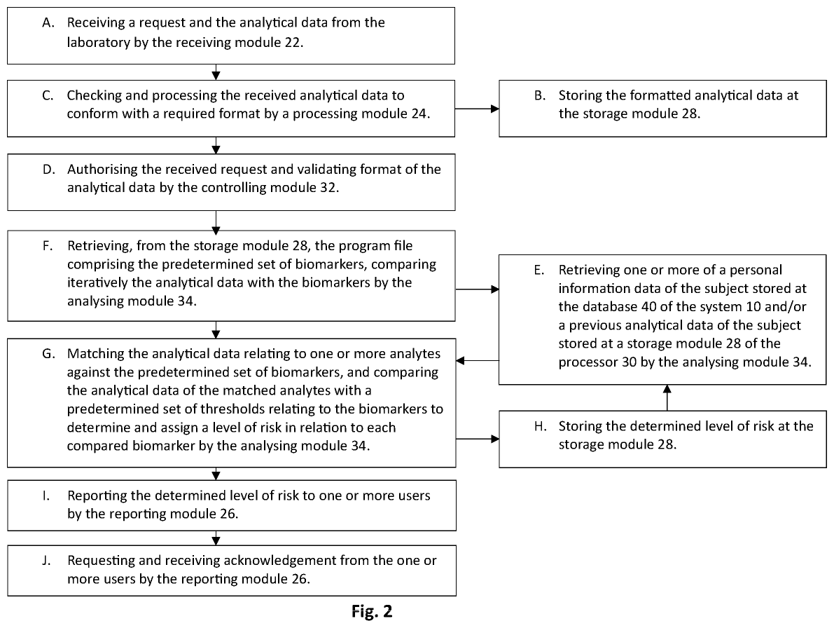

Particularly, the main unit 20 may comprise a receiving module 22 for receiving a request to evaluate a test result from the laboratory, the request including associated analytical data of the test result relating to one or more analytes identified from a biological sample such as a blood sample obtained from the subject (Step A, FIG. 2), and preferably also including data for the patient or subject. The analytical data may comprise test results from analysis performed based on one or more analytes which can be any biomolecules and/or compositions from the collected biological sample. In the context of the present invention, the biological sample may include one or more body fluids collected from the subject which can be blood, urine, vaginal discharge or secretion, post-surgery fluids, postpartum fluids, sweat, saliva, amniotic fluid, ascites, and semen, etc., and more preferably, a blood sample. Typically, the test subject or patient may, as requested by the subject's doctor, have their blood drawn at a laboratory or a testing facility by a healthcare professional. The healthcare professional may then follow the established protocols to ensure that the blood sample is collected safely. The drawn blood sample will be sent to the laboratory, where trained technicians will process the sample and perform the requested tests using specialized techniques and equipment. In one embodiment of the present invention, results of the blood tests will be sent and recorded directly by the laboratory via the main unit 20, with the results being received by the receiving module 22.

A processing module 24 of the main unit 20 may optionally be used to process the received analytical data, such as to check and to process the received analytical data to conform with a required format for further processing by the processor 30 (Step B, FIG. 2). The received analytical data and/or the formatted analytical data may optionally be stored at a storage module 28 at the main unit 20 (Step C, FIG. 2).

The system 10 further comprises a processor 30 for processing the received analytical data against a predetermined set of biomarkers to automatically determine an escalation level for the subject based on the biomarker data. The processor 30 can be an integrated part of the main unit 20 or an individual module separated from the main unit 20. In the embodiment as shown in FIG. 1, the processor 30 may comprise a controlling module 32 adapted to authenticate and authorize the received evaluation request and to validate the format of the analytical data (Step D, FIG. 2). The controlling module 32 may optionally determine whether the analytical data associated with the request may contain the predetermined biomarkers. After the request and the analytical data are validated, the analyzing module 34 of the processor 30 will then retrieve, such as from the storage module 28 of the main unit 20, a program file comprising a predetermined set of biomarkers and compare iteratively the received analytical data with the predetermined set of biomarkers (Step E, FIG. 2). In one embodiment, the analysing module 34 may perform matching of the analytical data relating to one or more analytes identified from the blood sample against the predetermined set of biomarkers, and subsequently, comparing the analytical data of the matched analytes with the predetermined set of thresholds relating to the biomarkers to thereby determine and assign a level of risk in relation to each compared biomarker (Step F, FIG. 2). The assigned levels of risk can be categorized into, but not limited to, escalation levels comprising: none/no, very low, low, medium, high, and very high risks in respect of each biomarker, which determine the potential course of action for test subject or the patient. For example, the test subject may be advised to consult their general practice doctor within 7 days if a medium level of risk is determined and reported; to consult their general practice doctor within 3 days if a high level of risk is determined and reported; and to seek immediate medical assistance within 24 hours if a very high level of risk is determined and reported. For example, in one specific embodiment, analytical data revealing a low-density lipoprotein (LDL) content of ≥4.9 mmol/L will be assigned to a category of low level of risk.

In one embodiment, the analysing module may further retrieve personal information data of the subject stored at a database 29 of the system 10 and/or previous analytical data of the subject stored at a storage module 28 of the main unit 20 of the system 10 (Step G, FIG. 2) if such data is not already included in the request. Preferably, the personal information data may comprise personal attributes such as, but not limited to, one or more of gender, age, weight, height, diet, blood type of the subject. The personal information data may include whether or not the subject is pregnant and, if pregnant, the stage or semester of the pregnancy.

One or more of the predetermined set of biomarkers can be processed in accordance with one or more of the personal attributes, such as the gender and/or the age of the subject. For example, in one specific embodiment, analytical data revealing a patient with an age of younger than 30 years old and having a cholesterol content of >7.5 mmol/L or a patient with an age of or older than 30 years old and having a cholesterol content of >9 mmol/L will be determined as having a low level of risk. In another embodiment, the predetermined thresholds relating to one or more biomarkers can be processed in view of the stored previous analytical data of the subject. For example, a male subject with an age of or younger than 60 years old having a ferritin content of >400 μg/L or <1000 μg/L with a previous test result of >400 μg/L or <1000 μg/L, respectively, will be determined as having a low level of risk.

In one embodiment, haemoglobin for females may be assigned: a “low level” escalation level for a value greater or equal to 110 g/L but less than 115 g/L; a “medium level” escalation level for a value greater or equal to 100 g/L but less than 110 g/L; a “high level” escalation level for a value greater or equal to 85 g/L but less than 100 g/L or a value greater or equal to 190/L but less than 165 g/L; or a “very high level” escalation level for a value less 85 g/L or greater than 190 g/L. In a similar although contrasting manner, haemoglobin for males may be assigned: a “low level” escalation level for a value less than or equal to 185 g/L; a “medium level” escalation level for a value greater or equal to 100 g/L but less than 110 g/L; a “high level” escalation level for a value greater or equal to 190/L; or a “very high level” escalation level for a value less 85 g/L or greater than 190 g/L. Creatine kinase for females may be assigned: a “medium level” escalation level for a value greater than 200 U/L but less than or equal to 800 U/L; a “high level” escalation level for a value greater than 800 U/L but less than or equal to 5000 U/L; or a “very high level” escalation level for a value greater than 5000 U/L. In a similar although contrasting manner, creatine kinase for males may be assigned: a “medium level” escalation level for a value greater than 320 U/L but less than or equal to 1000 U/L; a “high level” escalation level for a value greater than 1000 U/L but less than or equal to 5000 U/L; or a “very high level” escalation level for a value greater than 5000 U/L.

It will be appreciated that there is very limited evidence to suggest any link between creatine and haemoglobin biomarkers, but each may individually be indicative of a health condition. Furthermore, there remains a possibility which requires further investigation or research that levels of both of these biomarkers may together be indicative of a health condition. The technical problem addressed by the invention is addressed in part in assigning escalation level scores or values from a common or consistent escalation level scale to thresholds of biomarkers which are quantitatively and qualitatively very different and where there may be little or no known linkage between the biomarkers. The resulting technical effect is an automated assessment of some or all of a plurality of disparate biomarkers in a biological sample to provide an overall escalation level which is indicative of the need or not for further health assessment or intervention by clinicians and is indicative of the urgency of the need for further health assessment or intervention by clinicians.

In one embodiment, the predetermined set of biomarkers may comprise, but are not limited to: low-density lipoprotein (LDL), high-density lipoprotein (HDL), non-HDL cholesterol, cholesterol, triglyceride, total cholesterol/HDL ratio, triglyceride/HDL ratio, apolipoprotein A1 (APOA), apolipoprotein B (APOB), APOB/APOA ratio, total iron binding capacity, unsaturated iron binding capacity, ferritin, iron, transferrin saturation, haemoglobin, mean corpuscular haemoglobin, mean corpuscular volume, red blood cell count, basophil, monocyte, white blood cell, neutrophil, lymphocyte, platelet count, red blood cell distribution width, haematocrit, eosinophils, total protein, albumin, globulin, alanine aminotransferase (ALT), alkaline phosphatase (ALP), gamma-glutamyl-transferase (GGT), bilirubin, aspartate aminotransferase (AST), lipase, amylase, free triiodothyronine (FT3), thyroglobulin antibody, thyroid peroxidase antibody, total thyroxine (T4), thyroid-stimulating hormone, free thyroxine (FT4), reverse triiodothyronine (T3), total T3, high-sensitivity C-reactive protein (hsCRP), haemoglobin A1C (HbA1c), glucose, sodium, creatinine, estimated glomerular filtration rate (eGFR), urea, human chorionic gonadotropin (HCG), luteinizing hormone (LH), oestradiol, follicle-stimulating hormone (FSH), free testosterone, total testosterone, sex hormone-binding globulin (SHBG), free androgen index (FAI), prolactin, dehydroepiandrosterone sulfate (DHEAS), progesterone, anti-mullerian hormone (AMH), cortisol, C-peptide, creatine kinase, active vitamin B12, total vitamin B12, folate, omega 3, omega ration arachidonic (ARA)/eicosapentaenoic (EPA), vitamin D, chromium, magnesium, manganese, selenium, copper, B-type natriuretic peptide (NTpro BNP), uric acid, prostate-specific antigen (PSA), anti-tissue transglutaminase IgA (TTG-IgA), total IgA, anti-gliadin IgG, endomysial IgA, glucose-6-phosphate dehydrogenase (G6PD), chlamydia, gonorrhea, hepatitis B surface antibody (Anti-HBs), hepatitis B core antibody, hepatitis B surface antigen, human immunodeficiency virus (HIV), syphilis antibody, hepatitis C core antigen, urine albumin-creatinine ratio (uACR), varicella zoster antibody (IgG), measles antibody (IgG), mumps antibody (IgG), rubella antibody (IgG) and urine dip. More preferably, the biomarkers may relate to or comprise one or more of a quantity, concentration, ratio, distribution width, binding capacity, saturation and/or volume of one or more selected analytes.

The determined and assigned level of risk for each biomarker may be stored at the storage module 28 (Step H, FIG. 2). There is no essential need for the device 40 to store escalation result data. Such data is preferably stored elsewhere in the system 10 such as the storage module 28 and/or the database 29.

For a determined level of risk which requires immediate attention from the users, such as for certain level of risks of selected biomarkers based on a preset notification policy, the analysing module 34 of the processor 30 will instruct the reporting module 26 of the main unit 20 to report, via designated user interfaces, to the users whose attention is required, which can be the test subject or patient, the clinic or hospital which initiated the evaluation request, and/or the responsible doctor of the patient such as when the assigned risk level is very high and that an immediate or early medical treatment or assistance is required. The level of risk will be reported via the reporting module 26 to the required recipients. In one embodiment, the reporting module 26 may request an acknowledgement from the recipients to ensure that the recipients have been made aware of the determined level of risk, especially for the situation where immediate medical or early attention to the test subject is required.

In another aspect of the present invention, the device 40 comprises a processor 30 as described above which is adapted to process received analytical data, such as a test result received from a laboratory. The test result preferably relates to one or more analytes identified from a biological sample, such as a blood sample, obtained from the subject. The analytical data of the test result is then processed against a predetermined set of biomarkers to determine a risk or escalation level for the subject. Preferably, the analysing module 34 of the processor 30 performs matching of the analytical data relating to one or more analytes against the predetermined set of biomarkers, and subsequently, comparing the analytical data of the matched analytes with a predetermined set of thresholds relating to the biomarkers to thereby determine and assign a level of risk (escalation level) in relation to each compared biomarker. The assigned levels of risk can be categorized into, but not limited to, none/no, very low, low, medium, high, and very high risks in respect of each biomarker, which determine the potential course of actions the clinician or doctor may recommend to the test subject or the patient.

More specifically, one embodiment of the computer implemented method in accordance with the invention involves receiving analytical data relating to a plurality of analytes identified from a biological sample obtained from the subject. Then, processing the received analytical data against a predetermined set of biomarkers to automatically determine a level of risk for the subject. The processing step is preferably achieved by: retrieving a rule for each of at least some of the biomarkers identified in the analytical data, each rule defining a set of thresholds corresponding to some or all of a set of escalation levels, the escalation levels being indicative of different respective levels of risk to the subject. Then, comparing values of the at least some of the biomarkers to the thresholds defined by their respective rules to determine respective escalation levels for each of said at least some of the biomarkers. Finally, determining, selecting, or calculating an overall escalation level based on the determined respective escalation levels for each of said at least some of the biomarkers.

The set of escalation levels is common to all rules such that, whilst each rule defines a set of one or more thresholds for one or more of the escalation levels, the defined thresholds for each rule being materially specific to that rule and typically not interchangeable between rules, the use of a common set of escalation levels associated by each rule with one or more its respective thresholds allows an overall escalation level to be obtained automatically. The use of the common set of escalation levels prevents conflicts between different results for different biomarkers preventing an overall escalation level to be determined, selected, or calculated. Consequently, the method enables a plurality of different biomarkers to be assessed against the defined or preset thresholds of the specific rules and for an overall escalation level result to be automatically obtained to provide healthcare professionals and/or users or other parties with an indication of how soon a subject should be examined and assessed for any of one or more medical conditions.

It will be understood that the set of escalation levels could comprise a set of escalation values.

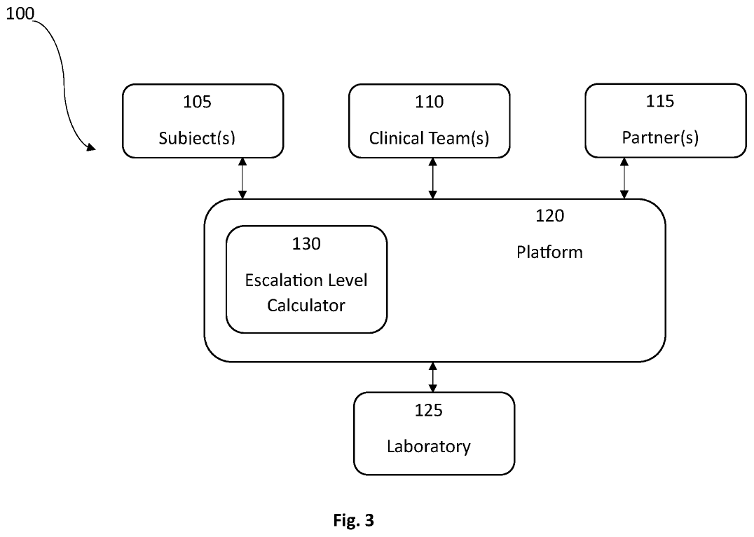

The general scenario in which the method of the invention is implemented is illustrated in FIG. 3. The illustrated system 100 includes the subject 105 for whom a blood test or the like has been performed. The clinical team 110 comprises the team responsible for overseeing the risk level information flow, i.e., escalation level information flow. The partner 115 may comprise a person or partner organization who requested the blood test be conducted on the subject 105 and who has ultimate responsibility for notifying subjects 105 about escalation risk levels. The platform 120 is an automated software-based system which processes, for example, blood test results (analytical data) and which notifies and displays results and escalation levels to relevant parties. The laboratory 125 conducts the blood test and provides the results to the platform 120. The escalation level calculator 130 comprises an automated software-based module which performs the steps of the method of the invention in determining, selecting, or calculating an escalation risk level for a subject 105 based on the test results and one or both of their personal attributes and previous test and medical history.

The system 100 may comprise a web-based system or any suitable data communication system for connecting the parties. The subject 105 may view results through a login service over a user interface provided by the platform 120. The clinical team 110 may use a secure link such as a virtual private network link or a dedicated network link to securely communicate with the platform and protect/secure the data. The partner 115 may communicate with the platform 120 via an application programming interface (API) and/or webhooks. The laboratory 125 and the escalation level calculator 130 preferably also connect to the platform 120 via suitable secure links in a like manner to the clinical team's connection to the platform 120.

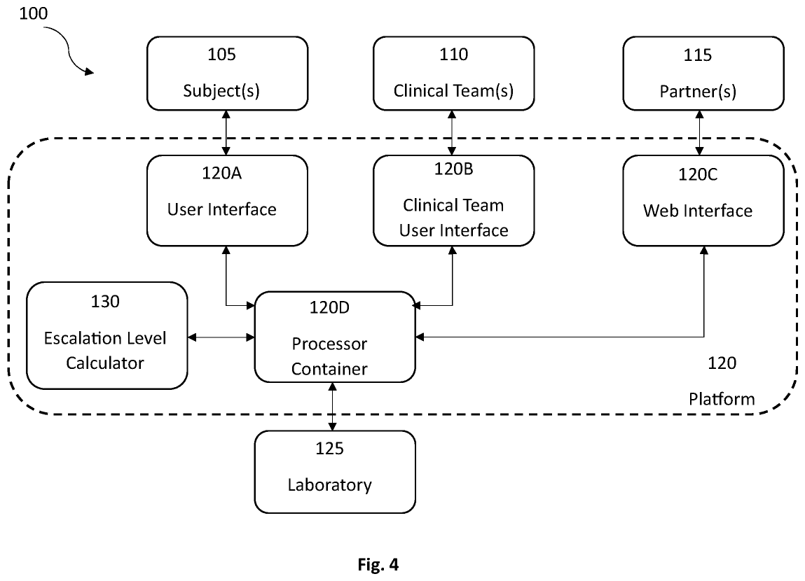

FIG. 4 provides a more detailed view of the system 100. The user interface 120A used by the subject 105 to connect to the platform 120 preferably comprises a vue.js container based on a JavaScript framework which enables the subject to login to the platform 120 to see results including escalation levels and to acknowledge receipt of an overall escalation level result. The vue.js container is preferably utilized because the containers enable machine code to be modularized by separating the presentation logic from the business logic which makes it easier to manage and maintain the application over time. Furthermore, containers can be reused across multiple components which reduces code duplication and makes it easier to scale the application. Containers also separate the components and the application state such that it is easier to manage the application and reduce risks of data conflicts. Containers also improve the performance of the application by optimizing the rendering process. The clinical team user interface 120B is also preferably comprises a vue.js container which enables the clinical team 110 to securely communicate with the platform 120 and to protect/secure the data. The processor container 120D preferably comprises a Ruby on Rails (RoR) container which is based on a web framework using a Model-View-Controller (MVC) architecture which packages and deploys applications in a scalable manner on different platforms. The RoR containers can be scaled horizontally across multiple servers to improve performance of the application and handle increasing traffic loads. Furthermore, RoR containers provide a consistent environment for the application to run which reduces the risk of compatibility issues. RoR containers can be deployed more rapidly as they do not require complex installation and configuration processes. The processor container 120D preferably parses and processes biological sample data from the laboratory 125, stores escalation level results, and tracks notifications and acknowledgements. The processor container 120D preferably also polls results continuously or periodically from a store of results. Periodical polling may be set for every minute. The store of results may form part of the platform 120 and/or may be hosted by the laboratory 125. The escalation level calculator 130 also preferably comprises a Ruby on Rails container which is configured to provide escalation level results for each set of analytical data (biological sample results). Preferably, the escalation level calculator 130 stores the biomarker rules as YML files. These files are formatted in accordance with the YAML Ain't Markup Language which are saved in plain text format and are appended with the “.yml” extension. Using YML files provides a number of advantages in that YML files are typically much shorter than equivalent XML or JSON files as they do not require as many characters for markup which makes them easier to manage and maintain. Furthermore, YML supports complex data structures including arrays, dictionaries and nested structures which makes it useful for a variety of applications from configuration files to data exchange. YML also supports comments which are used to provide additional context or explanations for the data in the files which allows users to understand the purpose of the data. YML is also a platform-independent format which means that the YML files can be used on any platform that supports the format making it easier to share data between different systems and platforms.

For each biological sample result received at the processor container 120D, the processor container 120D sends a request to the escalation level calculator 130 and subsequently stores and processes the escalation level results received in response from the escalation level calculator 130. The web interface 120C preferably comprises a Ruby on Rails container which is configured to provide a notification microservice which triggers, for example, emails or webhooks to inform relevant parties of escalation level results or alerts. The processor container 120D is configured to make calls to the web interface 120C when any relevant party needs to be notified of an escalation level results or alert. The web interface 120C conveys to the processor container 120D acknowledgements of receipt of escalation level results or alerts from relevant parties. The laboratory 125 preferably comprises an AWS S3 bucket. An AWS S3 bucket comprises a public cloud storage resource available in Amazon Web Services (AWS) Simple Storage Service (S3) Platform which provides object storage in S3 buckets in distinct units called objects instead of files. A bucket is a logical container of objects which comprises data, key (assigned name), and metadata. An AWS S3 bucket is highly scalable and can store almost unlimited amounts of data which is ideal for systems managing and handling large amounts of data. The AWS S3 bucket is durable in that multiple copies of the data are stored across multiple availability zones such that the data is always available and protected against data loss. Furthermore, AWS S3 buckets can be accessed from anywhere globally. In addition, the AWS S3 bucket includes several security features including encryption, access controls, and audit logs to defend against unauthorized access. AWS S3 buckets are easily integrated with other cloud based services. The laboratory 125 acts as an interface between the platform 120 and the real, physical laboratory which enables the laboratory to upload biological sample results to their designated S3 bucket.

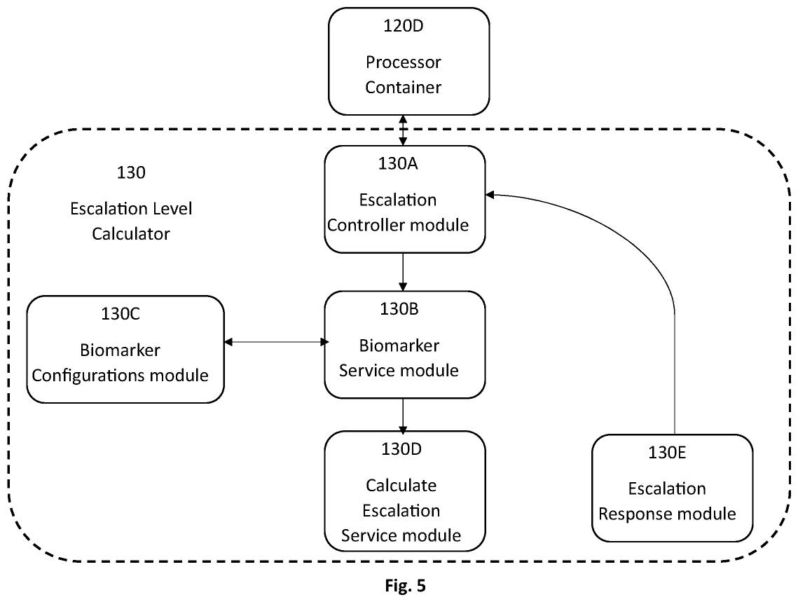

FIG. 5 provides a more detailed view of the escalation level calculator 130. The escalation controller module 130A authenticates and authorizes a party requesting an escalation level result or alert, validates that the received request is in accordance with a pre-determined format and that the request data contain valid biomarkers. The biomarker service module 130B loads respective rules from a set of stored rules for each valid biomarker in the request. The rules may be grouped in suitable classes ready for transferring to the calculate escalation service module 130D. The biomarker service module 130B loads the rules from the biomarker configurations module 130C which holds YML files for every biomarker that the escalation level calculator 130 serves. The calculate escalation service module 130D loops over every biomarker in the request and matches the correct rule for the biomarker or none if there is no match. The calculate escalation service module 130D then calculates escalation level results for each matched biomarker and subsequently determines, selects, or calculates an overall escalation result. Whilst the overall or final escalation level result may be calculated from all of the escalation level results for the matched biomarkers, the overall or final escalation result is preferably selected or determined as the highest escalation level category or value from the set of escalation level results. Alternatively, or additionally, one or more of the rules may designate that, if one or more of their defined thresholds are matched, the escalation level of the matched threshold must be selected as the overall or final escalation level result. This will typically be where the matched threshold is indicative of a more concerning health risk to the subject. Biomarkers may be grouped into weighted classes where the weight of the class is taken into account in determining or selecting an overall escalation level result. In one embodiment, the highest escalation level identified for any of the biomarkers is returned as the overall escalation level result. The escalation response module 130E stores the escalation level results for each biomarker, stores the rules that matched the biomarkers, and stores the overall escalation level result. It will be understood that of the modes 130A-E can be implemented as software modules.

The rules used by the escalation level calculator 130 to define escalations are stored in yaml files. These are static files that are controlled by the same version control that is used with all the source code. This provides benefits. First of all, by using yaml files stored in memory rather than a database, it means the rules are not changeable when a version of the escalation level calculator 130 has been deployed such that is always known which exact rules are in version “1.X.X” of the escalation level calculator 130. In the case of a database, the database could be updated or altered independently of the version of the escalation level calculator 130 thereby losing the connection to knowledge of the exact rules that were being used by the escalation level calculator 130. This improves the safety of the data, improves traceability such that, if someone had an escalation level calculated, determined, or selected using version “1.0.0” of the escalation level calculator 130, it would be known exactly which rules were used, and improves the speed of the device as no connections to databases for data retrieval need to be made. Preferably, the escalation level calculator 130 is not accessible via the public internet.

The escalation level report containing the overall escalation level result comprising an escalation notification or alert may be output to one or more of the subject, the subject's clinician, a pharmacist, a third-party health services provider, or a partner organization.

Each of the rules may not comprise a set of thresholds which match with all of the defined or possible escalation levels, i.e., there may be gaps in the set of thresholds compared to the set of defined or possible escalation levels. It is possible for a selected biomarker to have, for example, only one threshold which matches the highest escalation level value. In such a case, if the threshold is met by the biological sample results for that biomarker then the escalation level result for that biomarker will be the highest escalation level and this escalation level will then be returned as the overall escalation level result.

In one embodiment of the method implemented by the escalation level calculator 130, all the rules will be based alongside the results for the patient, and the method then loops through each biomarker using the following steps:
1. Load the biomarker and all its previous results;
2. Load the escalation rules for that biomarker;
3. Go to the ‘very high’ escalation level and load the rules for it;
4. Loop over each rule for the escalation level;
5. For each rule, loop over all the inclusion criteria for that rule;
   a. If all the inclusion criteria match, then the loop breaks and the escalation level for that rule is stored for that biomarker;
   b. If all the inclusion criteria do not match, it continues to loop;
6. After all the rules for an escalation level have been looped over, the escalation calculator 30 will then go down to the next level severity and return to step 4.

The order preferably goes: a. Very High; b. High; c. Medium; d. Low; e. Very Low;
7. If no rule is matched by the time the escalation level calculator 130 has reached the end of the rules for Very Low, then that biomarker is not escalated; and
8. The service will then move onto the next biomarker and continue until they have all been run through.

The escalation level calculator 130 is preferably configured such that requests comprising analytical data identifying biomarkers which do not form part of the set of defined biomarkers for which rules have been established are rejected. Counter-intuitively, this is advantageous as it prevents an overall or final escalation level being assigned to a request where undefined biomarkers are ignored as such biomarkers, if defined, might result in a higher escalation level result. It is safer that requests comprising analytical data identifying biomarkers which do not form part of the set of defined biomarkers are rejected as this negates or mitigates the possibility of ‘false positive’ or ‘false negative’ type escalation level results issuing from the escalation level calculator 130.

The escalation level calculator 130 is preferably also configured such that requests comprising analytical data identifying biomarkers with units not matching the units used in the established rules for such biomarkers are rejected. This is also advantageous as it prevents ‘false positive’ or ‘false negative’ type escalation level results issuing from the escalation level calculator 130 as a consequence of numerical values of incorrect units being taken as values for the correct units specified in the defined rules.

The overall or final escalation level result issued by the escalation level calculator 130 for a received request is traceable to the rule which triggered the overall or final escalation level result which has the advantage that, if an issued overall or final escalation level result is challenged or contested, the system 100 can trace how the issued overall or final escalation level result was determined, selected, or calculated by referring to the rules relating to the identified biomarkers in the request.

It is not an intention of the escalation level calculator 130 or system 100 to be used in an acute medical setting or to diagnose any medical conditions as such. The escalation level calculator 130 or system 100 is not directed at processing or analyzing test data for acute disease specific biomarkers which would typically require immediate clinical assessment and follow-up, although it is capable of doing so when appropriately configured. However, the focus of the escalation level calculator 130 or system 100 is processing and analyzing postal pathology samples with the intention to be able to report the overall or final escalation result to the or each end-user or relevant person a minimum of about 24 hours after the biological sample is taken. Consequently, the escalation level calculator 130 or system 100 is not intended for users seeking emergency medical services and not intended for users seeking acute medical diagnoses.

The combination of RoR containers, vue.js containers, and AWS S3 buckets provides a significant number of technical advantages including scalability, portability, consistency, security, and integration. RoR and vue.js containers can be easily scaled together horizontally to handle increasing traffic loads while AWS S3 buckets provide almost unlimited storage capacity. RoR and vue.js containers can be easily implemented and moved between different environments whilst AWS S3 buckets can be accessed from anywhere in the world. RoR and vue.js containers provide a consistent environment for the application to run. RoR and vue.js containers together with AWS S3 buckets can be readily integrated to provide a cost effective cloud-based system for a web delivered service which is highly scalable.

In the present invention, all of the relevant parties can access the biological sample data and the results data according to their respective data access privileges in a secure and data efficient manner.

Using a single escalation level result in the manner defined by the present invention covering some or all of a plurality of biomarkers in a biological sample provides several advantages including reducing the number of tests to be performed which may save time and resources for both patients and healthcare providers. Furthermore, using a single escalation level result in the manner defined enables a standardized approach to testing enabling test to be performed consistently and accurately which is typically not the case where different human clinicians are involved in assessing determining tests and in assessing biological sample results. Other advantages include a reduced risk of errors in results analysis and providing faster and more accurate diagnoses leading to earlier treatment and better outcomes for patients.

The present invention is advantageous in that it automates the review and evaluation processes on the test results in contrast to the conventional requirement of manual review and interpretation by medical doctors or clinicians. The present invention therefore negates the requirement for the medical doctors or clinicians to manually review all unusual or abnormal test results. The system may further automatically inform users whether follow-up consultation within a certain time-frame, or an immediate medical assistance or treatment, is required. The users can therefore be timely informed and alerted once any unusual or abnormal results are revealed by the tests, such that follow-up medical actions should be implemented early or without delay.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or other embodiments not recited herein. Accordingly, the invention also provides embodiments that comprise combinations of one or more of the illustrative embodiments described above. Modifications and variations of the invention as herein set forth can be made without departing from the spirit and scope thereof. Therefore, only such limitations should be imposed as indicated by the appended claims.

In the claims hereof, any element expressed as a means for performing a specified function is intended to encompass any way of performing that function. The invention as defined by such claims resides in the fact that the functionalities provided by the various recited means are combined and brought together in the manner the claims call for. It is thus regarded that any means that can provide those functionalities are equivalent to those shown herein.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A computer implemented method of automatically evaluating biomarkers in a biological sample taken from a subject, comprising the steps of:
receiving analytical data relating to a plurality of analytes identified from the biological sample; and
processing the received analytical data against a predetermined set of biomarkers to automatically determine an escalation level for the subject by:
retrieving a rule for each of the biomarkers identified in the analytical data, each rule defining a set of thresholds corresponding to some or all of a set of escalation levels, the escalation levels being indicative of different respective levels of a need to escalate the results;
comparing values of said biomarkers to the thresholds defined by their respective rules to determine respective escalation levels for each of said biomarkers; and
determining, selecting, or calculating an overall or final escalation level based on the determined respective escalation levels for each of said biomarkers.

2. The method of claim 1, wherein the method includes outputting the overall escalation level to automatically generate an escalation level report.

3. The method of claim 2, wherein the escalation level report is output to one or more of the subject, the subject's clinician, a pharmacist, a third-party health services provider, or a partner organization.

4. The method of claim 1, wherein the method includes continuously or periodically polling to retrieve analytical datasets relating to other subjects' biological samples and repeating the steps of the method of claim 1 for each retrieved analytical dataset.

5. The method of claim 1, wherein some of the rules do not define all of the set of escalation levels and/or some of the rules have gaps in the set of escalation levels.

6. The method of claim 5, wherein the step of comparing values of said at least some of the biomarkers to the thresholds defined by their respective rules comprises initially matching a rule from a database or store of rules for each of said at least some of the biomarkers or returning a null value where no matching rule is found.

7. The method of claim 1, wherein the method includes storing as an associated set of data: the rules retrieved for said at least some of the biomarkers; the respective escalation levels determined for said at least some of the biomarkers; and the overall escalation level.

8. The method of claim 1, wherein, prior to retrieving a rule for each of at least some of the biomarkers, the method includes processing the retrieved analytical data to determine that the analytical data is ordered in accordance with a predetermined format and contains valid biomarkers.

9. The method according to claim 1, further comprising the steps of retrieving or receiving one or more data selected from:
personal information data of the subject; and/or
stored previous analytical data of the subject.

10. The method according to claim 9, wherein the personal information data comprises one or more personal attributes selected from the group consisting of gender, age, weight, height, diet, blood type of the subject.

11. The method according to claim 9, wherein the one or more biomarkers are processed based on the one or more personal attributes.

12. The method according to claim 9, wherein the retrieved rules relating to the biomarkers are processed based on the stored previous analytical data of the subject.

13. The method according to claim 1, wherein the biomarkers are selected from the group consisting of: low-density lipoprotein (LDL), high-density lipoprotein (HDL), non-HDL cholesterol, cholesterol, triglyceride, total cholesterol/HDL ratio, triglyceride/HDL ratio, apolipoprotein A1 (APOA), apolipoprotein B (APOB), APOB/APOA ratio, total iron binding capacity, unsaturated iron binding capacity, ferritin, iron, transferrin saturation, hemoglobin, mean corpuscular hemoglobin, mean corpuscular volume, red blood cell count, basophil, monocyte, white blood cell, neutrophil, lymphocyte, platelet count, red blood cell distribution width, haematocrit, eosinophils, total protein, albumin, globulin, alanine aminotransferase (ALT), alkaline phosphatase (ALP), gamma-glutamyl-transferase (GGT), bilirubin, aspartate aminotransferase (AST), lipase, amylase, free triiodothyronine (FT3), thyroglobulin antibody, thyroid peroxidase antibody, total thyroxine (T4), thyroid-stimulating hormone, free thyroxine (FT4), reverse triiodothyronine (T3), total T3, high-sensitivity C-reactive protein (hsCRP), hemoglobin A1C (HbA1c), glucose, sodium, creatinine, estimated glomerular filtration rate (eGFR), urea, human chorionic gonadotropin (HCG), luteinizing hormone (LH), oestradiol, follicle-stimulating hormone (FSH), free testosterone, total testosterone, sex hormone-binding globulin (SHBG), free androgen index (FAI), prolactin, dehydroepiandrosterone sulfate (DHEAS), progesterone, anti-mullerian hormone (AMH), cortisol, C-peptide, creatine kinase, active vitamin B12, total vitamin B12, folate, omega 3, omega ration arachidonic (ARA)/eicosapentaenoic (EPA), vitamin D, chromium, magnesium, manganese, selenium, copper, B-type natriuretic peptide (NTpro BNP), uric acid, prostate-specific antigen (PSA), anti-tissue transglutaminase IgA (TTG-IgA), total IgA, anti-gliadin IgG, endomysial IgA, glucose-6-phosphate dehydrogenase (G6PD), chlamydia, gonorrhea, hepatitis B surface antibody (Anti-HBs), hepatitis B core antibody, hepatitis B surface antigen, human immunodeficiency virus (HIV), syphilis antibody, hepatitis C core antigen, urine albumin-creatinine ratio (uACR), varicella zoster antibody (IgG), measles antibody (IgG), mumps antibody (IgG), rubella antibody (IgG) and urine dip.

14. The method according to claim 1, wherein the rules relating to the biomarkers comprise thresholds based on one or more of a quantity, concentration, ratio, distribution width, binding capacity, saturation and/or volume of one or more selected analytes.

15. The method of claim 1, wherein the rules relating to the biomarkers include associating thresholds from the set of defined thresholds for that rule to respective thresholds of said defined set of thresholds.

16. The method of claim 1, wherein the set of escalation levels is common to all of the rules relating to the biomarkers.

17. A system for evaluating biomarkers in a biological sample taken from a subject, the system comprising:
a receiving module for receiving analytical data relating to a plurality of analytes identified from the biological sample; and
a processor for processing the received analytical data against a predetermined set of biomarkers to thereby automatically determine an escalation level for the subject by:
retrieving a rule for each of the biomarkers identified in the analytical data, each rule defining a set of thresholds corresponding to some or all of a set of escalation levels, the escalation levels being indicative of different respective levels of a need to escalate the results;
comparing values of said biomarkers to the thresholds defined by their respective rules to determine respective escalation levels for each of said biomarkers; and
determining, selecting, or calculating an overall or final escalation level based on the determined respective escalation levels for each of said biomarkers.

18. The system according to claim 17, wherein the receiving module and the processor operate independently at separate devices.

19. A device for evaluating biomarkers in a biological sample taken from a subject, comprising a processor configured to implement the steps of:
receiving analytical data relating to a plurality of analytes identified from the biological sample; and
processing the received analytical data against a predetermined set of biomarkers to automatically determine a need to escalate the results by:
retrieving a rule for each of the biomarkers identified in the analytical data, each rule defining some or all of a set of escalation levels, the escalation levels being indicative of different respective levels of the need to escalate the results;
comparing values of said biomarkers to their respective rules to determine respective escalation levels for each of said biomarkers; and
determining, selecting, or calculating an overall or final escalation level based on the determined respective escalation levels for each of said biomarkers.
